(19) United States
(12) Patent Application Publication
(10) Pub. No.: US 2010/0222727 A1
(43) Pub. Date: Sep. 2, 2010

(54) EXOPROSTHESIS OR ORTHESIS FOR STABILIZING THE HUMAN KNEE JOINT

(75) Inventor: Hans Naegerl, Gleichen (DE)

Correspondence Address: LEYDIG, VOIT AND MAYER, TWO PRUDENTIAL PLAZA, SUITE 4900, 180 NORTH STETSON AVENUE, CHICAGO, IL, 60601, US

(73) Assignee: AEQUOS Endoprothetik GmbH, Graefelfing (DE)

(21) Appl. No.: 12/294,916

(22) PCT Filed: Mar. 19, 2007
(86) PCT No.: PCT/DE07/00518

§ 371 Date: Sep. 26, 2008

(30) Foreign Application Priority Data
Mar. 28, 2006 (DE) 20 2006 005 095.7

(51) Int. Cl. A61F 5/00

(52) U.S. Cl. 602/16

(57) ABSTRACT

A device configured in the form of an exoprosthesis or orthosis for stabilizing a human knee includes a femur part associated with a thigh and configured to be affixed to the thigh a tibia part associated with a lower leg and configured to be affixed to the lower leg, and a joint connecting the femur part to the tibia part. The joint is configured so that, during a flexing motion of the knee from a straight position to a flexed position, an instantaneous center of rotation of the joint moves along a curved centrode of the tibia having a vectorial vertical component and a vectorial sagittal component, the vectorial saggital component runs from posterior to anterior relative to the femur part.

This application is a U.S. National Phase application under 35 U.S.C. §371 of International Application No. PCT/DE2007/000518, filed on Mar. 19, 2007, which claims priority to German Patent Application No. DE 10 2006 005 095.7, filed on Mar. 28, 2006. The International Application was published in German on Oct. 4, 2007 as WO 2007/110050 under PCT Article 21(2).

The present invention relates to a device configured in the form of an exoprosthesis or orthosis for stabilizing the human knee joint, comprising a femur part that is associated with the thigh and that can be affixed to the thigh, and comprising a tibia part that is associated with the lower leg and that can be affixed to the lower leg, the femur part and the tibia part being connected to each other by a joint by means of which, during the flexing motion of the knee joint out of a straight position into a flexed position, the instantaneous center of rotation of the joint moves along a curved centrode comprising a vectorial vertical component as well as a vectorial sagittal component.

### BACKGROUND

In order to support the natural range of motion, DE 689 28 511 T2 proposes a multiaxis controlled-motion knee orthosis employing a sliding guide. For this purpose, a joint mechanism is provided that has two recesses configured as camming slots and two guide elements configured as cam pin followers, which are supposed to match the biomechanical concepts. Here, one of the camming slots is disposed in a transverse plane and serves to allow the forward motion of the femur part, while the second camming slot is disposed in a longitudinal orientation and defines a long arc segment for a unicentric phase of the joint arthrokinematics. During an initial range of the motion, pivoting occurs through a short transverse arc segment about an upper cam pin follower disposed within the longitudinally extending arcuate slot. After the lower cam pin follower reaches the anterior end of the transverse slot, the lower cam pin follower serves as the axis of rotation or pivot point for the motion of the upper cam pin follower along the length of the long arc segment of the longitudinal slot. The natural range of motion is supposed to be replicated in that the joint produces an initial rearward motion of the tibia relative to the femur of approximately 8-9 millimeters through the first 25 degrees of the flexing motion.

In a similar manner, U.S. Pat. No. 4,773,404 A and EP 01 73 161 A describe a multiaxis controlled-motion knee orthosis. These orthoses each comprise a joint that is configured in such a way that the tibia is forced to slide relative to the femur for a certain distance during an initial range of flexion of the knee out of a straight leg position.

EP 04 82 809 A1 already describes an orthopedic or prosthetic joint consisting of a member system with a plurality of members and a spring member having a variable length, five members of fixed length being provided which are rotatably connected to each other in a geometric arrangement in such a way that the spring member affixes the joint in every angular position of the various pivot-angle positions. In particular, the spring member blocks the joint in every angular position in such a way that the joint can only be curved by release means.

DE-PS 412 362 relates to an artificial knee joint for supporting the natural knee joint, having two axes of rotation that pass through an interconnecting piece that is articulated on two rails. The interconnecting piece is mounted between the inwards-facing flat sides of the superimposed ends of the two rails. The lower rail is articulated onto the interconnecting piece by the upper pivot of the two pivots while the upper rail is articulated onto the interconnecting piece by the lower pivot.

### SUMMARY OF THE INVENTION

An aspect of the present invention is to provide a considerably improved range of motion in a generic device configured as an exoprosthesis or orthosis for stabilizing the human knee joint. A further or alternate aspect is to replicate the range of motion of the natural joint motion to the greatest extent possible.

According to the invention, a device is provided in which the sagittal component of the centrode of the tibia part runs from posterior to anterior relative to the femur part during the flexing motion of the lower leg. In contrast to the prevailing opinion among experts which states that the device should execute an initial rearwards motion, especially a rearwards sliding of the tibia relative to the femur when the flexing motion is initiated—a range of motion that contradicts this opinion leads to a much more natural joint motion in actual practice. In fact, according to the invention, an initial forward motion of the tibia relative to the femur occurs, resulting in a purely rolling motion but not in a sliding of the knee joint.

A motion around a single pivot point does not correspond to the natural relative motion of the femur and the tibia, so that especially an exoprosthesis or orthosis constructed according to the principle of a hinged joint could lead to unsatisfactory results.

In this context, it has been found to be particularly conducive if the sagittal component of the centrode of the tibia part runs from posterior to anterior relative to the femur part exclusively during an initial angular range of the flexing motion of the lower leg. In this manner, the rolling motion is limited to an initial angular range so as to further optimize the range of motion.

Moreover, it has been found to be especially advantageous if the vertical component of the centrode of the tibia part runs from caudal to cranial relative to the femur part during the flexing motion of the lower leg, so as to achieve a further improvement and approximation of the natural joint motion.

The multi joint motion can fundamentally be attained by means of appropriately configured molded parts whose functional interaction defines the centrode. However, it is especially conducive to implement a modification of the present invention in which the joint has a sliding guide with two groove-shaped recesses, especially in the form of cutouts, that each accommodate a guide element, especially a projection, which is configured as a slot nut, whereby the sliding guide and the guide elements are each connected to the femur part or to the tibia part. In this manner, a very flat structure can be achieved, with the additional benefit that it is easy to manufacture. Furthermore, in order to reduce the friction resistance, the guide elements can be fitted with rollers that are held movably in the recess and that are also prone to very little wear and tear. Moreover, this version allows the transmission of large forces.

Fundamentally, in order to achieve the desired range of motion, two guide elements are needed that each engage with a groove-shaped recess. However, an especially practical modification is one in which the sliding guide has exactly three recesses that each accommodate a guide element. In this manner, especially in the extreme positions of the joint, a largely uniform motion is ensured which especially prevents the possibility of increased motion resistance due to an unfavorable relative position of the guide elements, which could lead to jamming. It is also conceivable to have an additional restoring element against whose restoring force the guide elements can be moved into the extreme positions.

Another especially simple refinement is achieved if one of the recesses has a flat design, especially parallel to the sagittal axis, and thus allows a simple determination and definition of the desired initial forward motion.

Here, it is especially practical if the recesses have a steady course, as a result of which a uniform joint motion is ensured. This is associated with a perceptible improvement in the range of motion for patients, and at the same time, it avoids the sensation of inadequate support, which is inevitable with the prior art.

Another modification that is likewise especially practical is achieved if the joint has inner teeth and an intermeshing gear wheel that are each non-rotatably connected to the femur part or to the tibia part. In this manner, the desired centrode course is achieved simply by means of the gear wheel that intermeshes with the inner teeth; said gear wheel is pivoted around its own center axis when the joint makes a pivoting motion so that, in turn, the center axis is moved around a centrode that is parallel to the rolling circle or to the rolling orbit of the inner teeth.

An especially advantageous embodiment of the present invention is one in which the device has a thigh fixation means, especially a thigh rail, connected to the femur part, as well as a lower leg fixation means, especially a lower leg rail, connected to the tibia part, as a result of which it allows a simple fixation of the exoprosthesis or orthosis.

### BRIEF DESCRIPTION OF THE DRAWINGS

The invention allows various embodiments. In order to further elucidate their basic principle, one of them is shown in the drawing and is described below. The drawing shows the following side views:

FIG. 1 a device according to the invention in an extended joint position;

FIG. 2 the device shown in FIG. 1 in a flexed joint position;

FIG. 3 another device according to the invention, with three guide elements in an extended joint position;

FIG. 4 the device shown in FIG. 3 in a flexed joint position;

FIG. 5 another device according to the invention, with a toothed gear in an extended joint position; and

FIG. 6 the device shown in FIG. 5 in a flexed joint position.

### DETAILED DESCRIPTION OF A PREFERRED EMBODIMENT

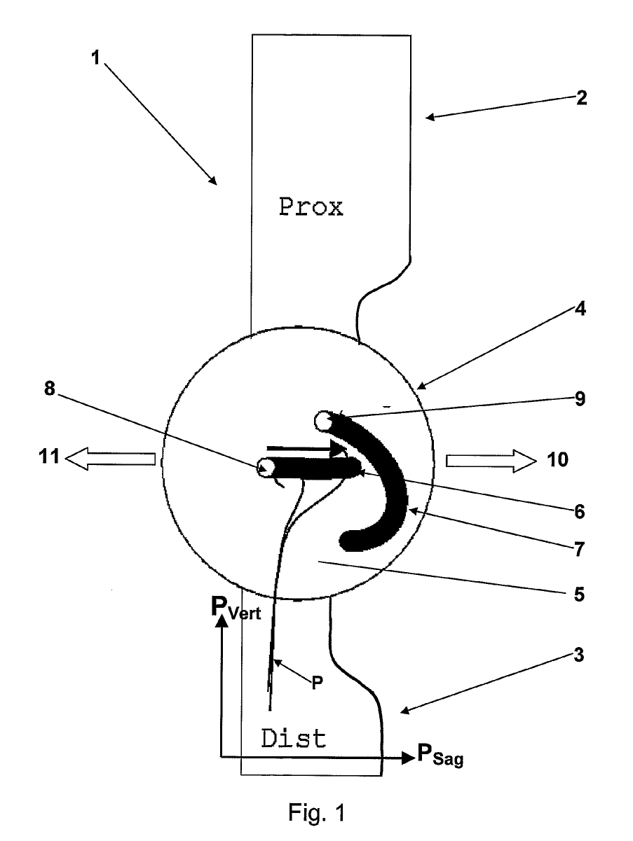

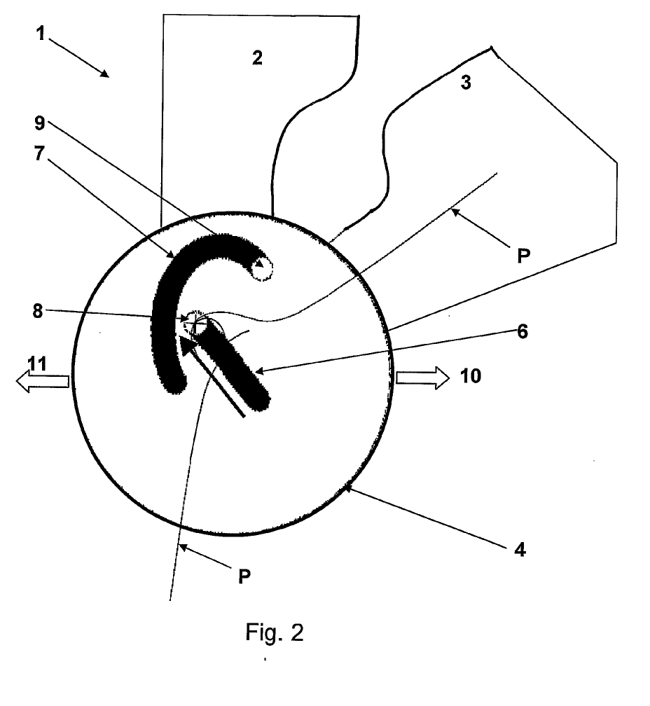

FIG. 1 shows a device 1 configured as an orthosis for stabilizing the human knee joint, with a femur part 2 that is intended for fixation to the thigh and with a tibia part 3 that is associated with the lower leg and that can be affixed to the lower leg, the femur part 2 and the tibia part 3 being connected to each other by a joint 4. The joint 4 has a sliding guide 5 that is connected to the tibia part 3 and that has groove-shaped recesses 6, 7 with which guide elements 8, 9 of the femur part 2 engage in order to generate a multi joint motion. As a result, during the flexing motion of the joint 4 of the knee joint out of the depicted straight position, the instantaneous center of rotation moves along a curved centrode P into a flexed position shown in FIG. 2. The sagittal component P_{sag} of this centrode of the tibia part 3 runs from posterior 10 to anterior 11 relative to the femur part 2, whereas the vertical component Pᵥₑᵣₜ of the centrode P of the tibia part 3 runs from caudal to cranial relative to the femur part 2 during the flexing motion of the lower leg. Therefore, in particular, a first guide element 8 moves into recesses 6 parallel to the sagittal axis during the flexing motion, from a posterior position depicted in FIG. 1 into an anterior position depicted in FIG. 2.

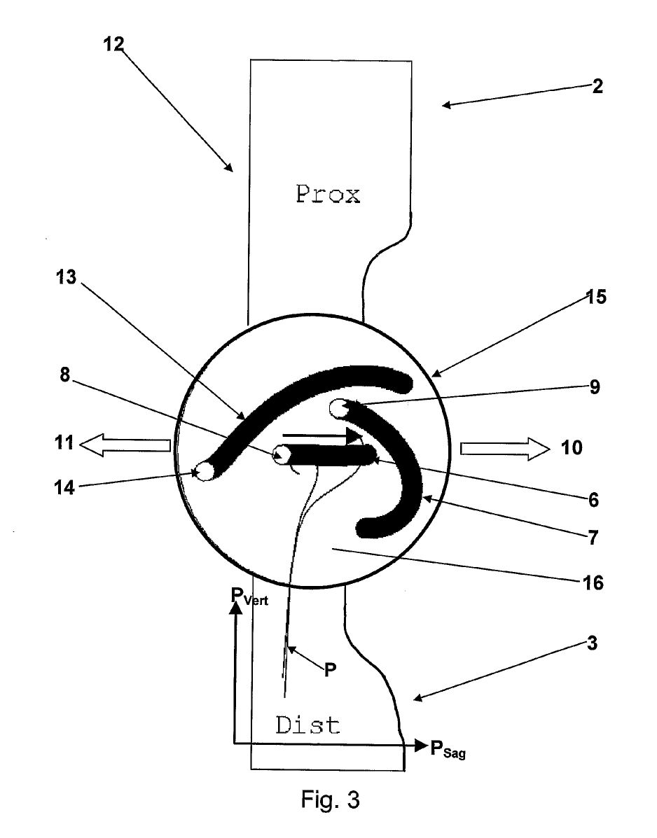

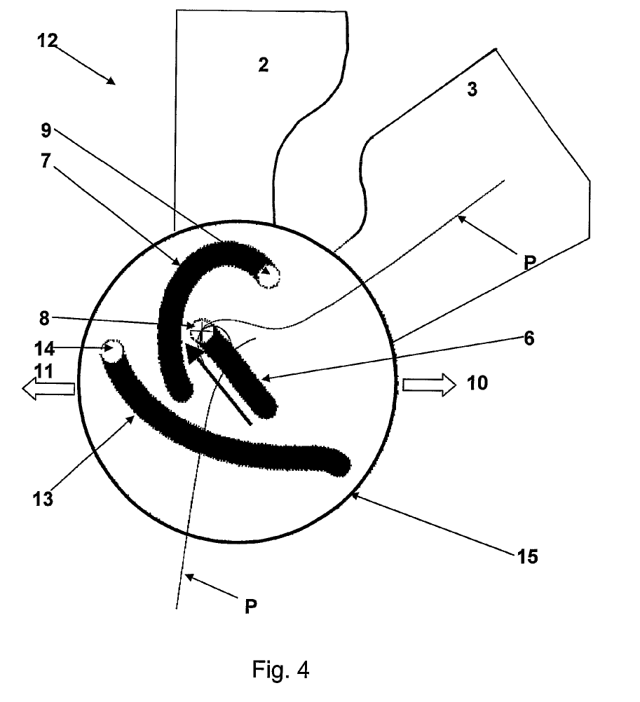

In contrast, FIGS. 3 and 4 show another device 12 according to the invention with an additional guide element 14 that engages with a recess 13 in an extended position or in a flexed joint position. By using three guide elements 8, 9, 14, the range of motion is further improved in terms of the joint resistance so that especially an unfavorable motion resistance is avoided and the resistance forces are altogether reduced. The femur part 2 that is associated with the thigh (not shown here) and that can be affixed to the thigh, as well as the tibia part 3 that is associated with the lower leg (not shown here) and that can be affixed to the lower leg are connected to each other by means of a joint 15. A sliding guide 16 of the joint 15 has precisely three recesses 6, 7, 13 that each accommodate a guide element 8, 9, 14 in order to generate the desired multi joint motion. During the flexing motion of the lower leg, the sagittal component P_{sag} of the centrode P of the tibia part 3 runs from posterior 10 to anterior 11 relative to the femur part 2 out of the extended position shown in FIG. 3 into a flexed position shown in FIG. 4.

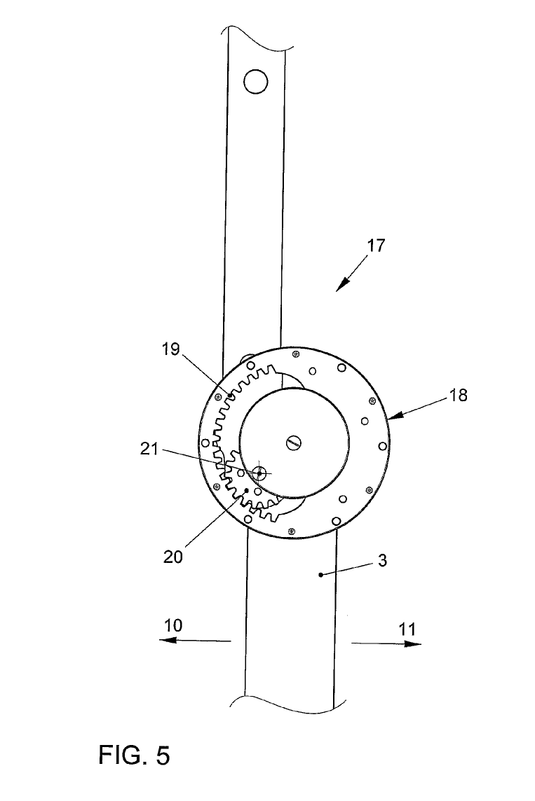

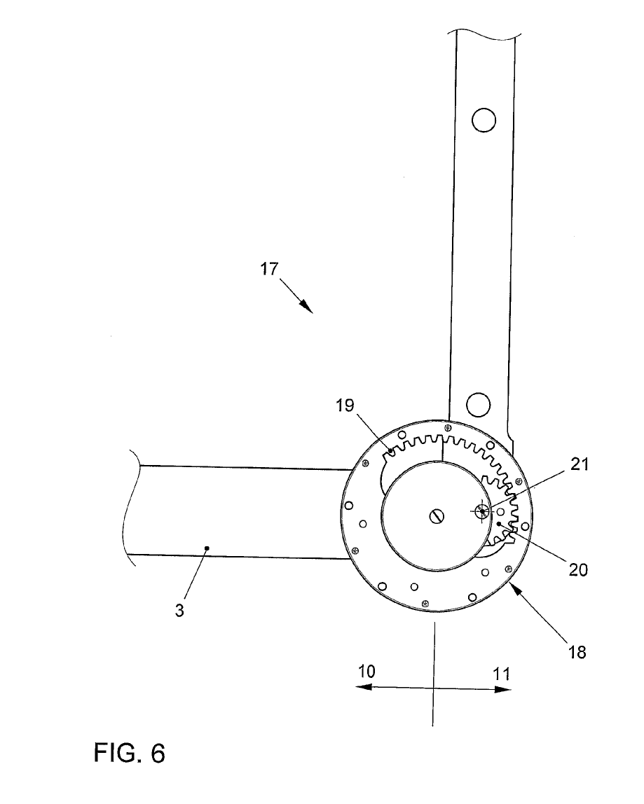

FIGS. 5 and 6 show another device 17 according to the invention with a joint 18 that has a toothed gear configured as inner teeth 19 and an intermeshing gear wheel 20. The gear wheel 20 that is non-rotatably connected to the femur part 2 engages with the inner teeth 19 connected to the tibia part 3. The pivoting motion of the joint 18 out of the extended position shown in FIG. 5 into the flexed joint position shown in FIG. 6—shown by a pivoting motion of the tibia part 3 from anterior 10 to posterior 11—leads to a rolling motion along the rolling circle. At the same time, a center axis 21 of the gear wheel 20 moves onto a centrode P that is parallel to the rolling circle.

1.-10. (canceled)

11. A device configured in the form of an exoprosthesis or orthosis for stabilizing a human knee, the device comprising:
a femur part associated with a thigh and configured to be affixed to the thigh;
a tibia part associated with a lower leg and configured to be affixed to the lower leg; and
a joint connecting the femur part to the tibia part and configured such that, during a flexing motion of the knee from a straight position to a flexed position, an instantaneous center of rotation of the joint moves along a curved centrode of the tibia having a vectorial vertical component and a vectorial sagittal component, the vectorial saggital component runs from posterior to anterior relative to the femur part.

12. The device as recited in claim 11, wherein the vectorial sagittal component runs from posterior to anterior relative to the femur part exclusively during an initial angular range of the flexing motion.

13. The device as recited in claim 11, wherein the vectorial vertical component of the centrode runs from caudal to cranial relative to the femur part during the flexing motion.

14. The device as recited in claim 11, wherein the joint includes a sliding guide.

15. The device as recited in claim 14, wherein the sliding guide includes two groove-shaped recesses each accommodating a guide element.

16. The device as recited in claim 15, wherein the sliding guide is fixed to one of the femur part and the tibia part and the guide elements are fixed to the other of the femur part and the tibia part.

17. The device as recited in claim 15, wherein the groove-shaped recesses include cutouts and the guide elements include slot nuts.

18. The device as recited in claim 15, further comprising a third groove-shaped recess accommodating a third guide element.

19. The device as recited in claim 15, wherein one of the two recesses is flat and oriented parallel to a sagittal axis.

20. The device as recited in claim 15, wherein the recesses form a continuous course.

21. The device as recited in claim 11, wherein the joint has includes inner teeth non-rotatably connected to one of the femur part and the tibia part and an intermeshing gear wheel non-rotatably connected to the other of the femur part and the tibia part.

22. The device as recited in claim 11, further comprising a thigh fixation device, connected to the femur part and lower leg fixation device connected to the tibia part.

23. The device as recited in claim 22, wherein the thigh fixation device includes a thigh rail.

24. The device as recited in claim 22, wherein the lower leg fixation device includes a lower leg rail.
